(19) United States
(12) Patent Application Publication
(10) Pub. No.: US 2017/0360681 A1
(43) Pub. Date: Dec. 21, 2017

(54) SKIN-PROTECTIVE EMULSIONS

(71) Applicant: KIMBERLY-CLARK WORLDWIDE, INC., Neenah, WI (US)

(72) Inventors: Stacy A. Mundschau, Weyauwega, WI (US); SangHa Park, Yongin-Si (KR); Corey T. Cunningham, Larsen, WI (US)

(21) Appl. No.: 15/533,777

(22) PCT Filed: Dec. 22, 2015
(86) PCT No.: PCT/US2015/067411

§ 371 Date: Jun. 7, 2017

Related U.S. Application Data
(60) Provisional application No. 62/096,143, filed on Dec. 23, 2014.

(51) Int. Cl. A61K 8/81; A61K 8/49; A61K 8/31; A61K 8/06; A61K 8/39; A61K 8/37; A61Q 17/00; A61K 8/92

(52) U.S. Cl. CPC A61K 8/8147; A61Q 17/00; A61K 8/4973; A61K 8/92; A61K 8/8111; A61K 8/062; A61K 8/39; A61K 8/375; A61K 8/31; A61K 2800/54

(57) ABSTRACT

Cosmetic oil-in-water emulsions that can provide a hydrophobic film to protect from excessive hydration are disclosed. In some aspects, a cosmetic oil-in-water emulsion can include water at a concentration of at least about 70% by total weight of the cosmetic oil-in-water emulsion, a plurality of water-in-oil emulsifiers, a base oil, an oil-in-water emulsifier, and a film-forming polymer. The base oil can have a wVTR of about 1.0 to 30 g/m²/hr and can be at a concentration of no more than about 15% by weight of the cosmetic oil-in water emulsion.

### TECHNICAL FIELD

The present invention relates to cosmetic oil-in-water emulsions for application to the skin, the emulsions having relatively low solids content and a low water-vapor transport rate.

### BACKGROUND OF THE DISCLOSURE

Occupational contact dermatitis (OCD) is an inflammatory response of the skin. OCD is commonly found on the hands after contact with water, detergents, soaps, solvents, food, oils, and the like. Typical symptoms of OCD range from redness, edema, vesiculation, thickening of the skin, hyperkeratosis, and scaling and fissuring. Itching, burning sensations, and cracks are the most common complaints, sometimes leading to pain and infection.

Although the prevalence of OCD varies widely based on occupation, it is listed as the most common of occupational disorders both in U.S and Europe. In 2010, the U.S. Bureau of Labor Statistics determined that OCD accounted for about 16.5 percent of all reported private industry occupational injuries and illness. Ultimately, OCD leads to employee absenteeism and loss of productivity. It is estimated that OCD accounts for $1.4 billion in direct medical expenses and another estimated $500 million in productivity losses. Despite these facts, OCD has been a long standing, largely unaddressed problem within industry despite recommended practices being widely available.

An entire regimen of personal protective products are necessary to address this problematic skin condition. For a number of industrial settings, it is advisable to use a formulation that protects against the two most common sources of irritation, detergents and excessive hydration. Hydrophobic and occlusive films provide resistance to irritation caused by the swelling of the corneocyte, and resistance to the penetration of irritating materials.

The hydrophobic or occlusive properties of a particular formulation may be determined by measuring water vapor transport rate (wVTR) and contact angle. Substances with a low wVTR and a high contact angle are highly occlusive and protective against excessive hydration when the skin is exposed to water-based materials for an extended period. Typical skin protectants use a high level of occlusive materials such as petrolatum to create a thick, substantive cream. This translates to higher material costs and poor aesthetics of the formulation (e.g., a greasy or sticky feeling), which can result in higher cost of products for consumers, and low product usage by employees. Additionally, skin protectants can even interfere with tasks common in industrial jobs. Ultimately, these factors can lead to lower overall usage or dosing of the formulation, lowering the actually efficacy provided to the user.

What is needed is a formulation that provides a very hydrophobic film to protect from excessive hydration while having low solids content and acceptable consumer aesthetics. Additionally, the hydrophobic film may be resistant to wash off in the presence of detergents.

### SUMMARY OF THE DISCLOSURE

In one embodiment, a cosmetic oil-in-water emulsion can include water at a concentration of at least about 70% by total weight of the cosmetic oil-in-water emulsion. The cosmetic oil-in-water emulsion can also include a plurality of water-in-oil emulsifiers and a base oil. The base oil can have a wVTR of about 1.0 to 30 g/m²/hr and can be at a concentration of no more than about 15% by total weight of the cosmetic oil-in water emulsion. The cosmetic oil-in water emulsion can additionally include an oil-in-water emulsifier and a film-forming polymer.

In another embodiment, a cosmetic oil-in-water emulsion can include water at a concentration of about 70% to about 95% by total weight of the cosmetic oil-in-water emulsion. The cosmetic oil-in-water emulsion can also include a base oil and an oil-in-water emulsifier. The cosmetic oil-in-water emulsion can further include at least two water-in-oil emulsifiers that are selected from the group consisting of: Diisostearoyl Polyglyceryl-3 Dimer Dilinoleate, Polyglyceryl-4 Diisostearate/Polyhydroxystearate/Sebacate, Polyglyceryl-2 Diisostearate, Polyglyceryl-2 Dipolyhydroxystearate, PEG-30 Dipolyhydroxystearate, Glyceryl Stearate, and combinations thereof. The cosmetic oil-in-water emulsion can further include a film forming polymer.

### DETAILED DESCRIPTION OF THE DISCLOSURE

The present disclosure relates to cosmetic oil-in-water emulsions that are breathable, have a relatively low wVTR, and have a low solids content. In some embodiments, the cosmetic oil-in-water emulsions include water at a concentration of about 70% to about 95% (by total weight of the cosmetic oil-in-water emulsion), at least one oil-in-water emulsifier, a plurality of water-in-oil emulsifiers, and at least one a film-forming polymer. The film-forming polymer can significantly lower the wVTR. Also, in some embodiments, the cosmetic oil-in-water emulsion of the present disclosure can include less than 15.0% (by total weight of the composition) of a material having a wVTR of about 1.0 to about 30.0 g/m²/hr. In some embodiments, the emulsions can have a pre-immersion wVTR value of about 1.0 to about 35.0 g/m²/hr. In some embodiments, the cosmetic oil-in-water emulsions can have a post-immersion wVTR value no greater than 40.0 g/m²/hr. Additionally, in some embodiments, the cosmetic oil-in-water emulsions can have an Oxygen Permeability of at least 1.0 cc/100 in² per day.

The emulsions of the current disclosure can provide the protection often achieved with water-in-oil emulsions, but have superior aesthetics as oil-in-water emulsions. Additionally, the relatively high water content of the cosmetic oil-in-water emulsions of the present disclosure makes the formulations less expensive to manufacture, and therefore more affordable for consumers. In addition, one of ordinary skill in the art will realize that such breathable emulsions with low solids content have better aesthetics and performance than Petrolatum or other highly occlusive compositions.

For purposes herein, “water-in-oil emulsions” are those in which the continuous, or external phase of the emulsion is oil and a pH measurement cannot be obtained. “Water-in-oil emulsifiers” are defined as having a hydrophilic-lipophilic balance (HLB) value less than 10.0. For purposes herein, “oil-in-water emulsions” are emulsions in which the external phase is water, allowing a pH measurement to be readily obtained. “Oil-in-water emulsions” of the present disclosure have no less than 70% water. “Oil-in-water emulsifiers” have an HLB value greater than or equal to 10.0.

For purposes herein, a “stable” emulsion is one that remained consistent in terms of appearance and having no oil/water phase separation after exposing the emulsion to three freeze-thaw cycles and three months at 40° C. Additionally, emulsions were exposed to an additional month at 40° C. testing and 50° C. testing as a means to determine whether differences existed among formulas and give greater confidence in their stability.

### Base Components

The base components of the cosmetic oil-in-water emulsion of the present disclosure include water, a base oil, an oil-in-water emulsifier, at least two water-in-oil emulsifiers, and a film forming polymer. As described above, the water concentration of the cosmetic oil-in-water emulsions of the present disclosure is at least about 70% by total weight of the oil-in-water emulsion formulation. In some embodiments, the water concentration can be between about 70% and about 95% by total weight of the oil-in-water emulsion. Further description regarding the base components of oil-in-water emulsifiers, water-in-oil emulsifiers, and film-forming polymers is presented below.

Base Oil:

The base oil can be any oil with a wVTR less than 30.0 g/m²/hr. In some embodiments, the base oil can have a wVTR of about 5.0 to about 30 g/m²/hr. In some embodiments, the base oil can provide a concentration of no more than about 15% of the cosmetic oil-in-water emulsion. Base oils can be, but are not limited to, Petrolatum, Mineral Oil, Lanolin, Hydrogenated Didecene, and Hydrogenated Polydecene.

Oil-in-Water Emulsifiers:

The cosmetic oil-in-water emulsion can include at least one oil-in-water emulsifier. As previously noted, oil-in-water emulsifiers have an HLB value greater than or equal to 10.0. In one aspect the primary emulsification system is at least one oil-in-water emulsifier, and in some embodiments, can be more than one oil-in-water emulsifier. In one embodiment, the primary emulsification system includes PEG-40 Stearate and Steareth-21. However, it is contemplated that other emulsification systems may be used. Non-limiting examples of alternative or additional oil-in-water emulsifiers include, but are not limited to, OLIVEM® 1000 (Cetearyl Olivate, Sorbitan Olivate; B&T Company s.r.l), PROLIPID® 151 (Stearic Acid, Behenyl Alcohol, Glyceryl Stearate, Stearyl Alcohol, Cetyl Alcohol, Palmitic Acid, Hydroxyethyl Cetearamidopropyldimonium Chloride and Myristyl Alcohol; Ashland Corporation), ARLACEL 165 (Glyceryl Stearate and PEG-100 Stearate; CRODA International) or MONTANOV 68 (Cetearyl Alcohol and Cetearyl Glucoside; SEPPIC Worldwide). Additionally, individual emulsifiers such as VARISOFT® PATC (Palmitamidopropyltrimonium Chloride; Evonik Industries), AMPHISOL® A (Cetyl Phosphate, DSM) CUTINA® GMS (Glyceryl Stearate; BASF), Myrj S40 (PEG-40 Stearate) or other emulsifiers may be used alone or in combination with those used for cosmetic purposes to create a stable emulsion. In some embodiments, the concentration of oil-in-water emulsifiers ranges from about 0.1% to about 10.0% (by total weight of the oil-in-water emulsion). In some preferred embodiments, the concentration of oil-in-water emulsifiers ranges from about 1.0% to about 5.0%, and even more preferably, from about 1.0% to about 3.0% (by total weight of the oil-in-water emulsion).

Water-in-Oil Emulsifiers:

In addition to the oil-in-water emulsification system, the oil-in-water emulsions of the present disclosure include a plurality (at least two) of water-in-oil emulsifiers. As noted above, water-in-oil emulsifiers have an HLB less than 10. In some embodiments, preferred water-in-oil emulsifiers have an HLB of 6 or less. Exemplary water-in-oil emulsifiers include, but are not limited to, Laureth-4, (Caprylic/Capric/Myristic/Stearic Triglyceride), (SOFTISAN® 378, Cremer Oleo), Polyglyceryl-2 Caprate (CREMERCOOR® PG2 C10, Cremer Oleo), Polyglyceryl-4 Cocoate (CREEMERCOOR® PG4 COCOATE, Cremer Oleo), Polyglyceryl-3 Distearate (CREMOPHOR® GS 32, Cremer Oleo), Sunflower Seed Oil Sorbitol Esters (HOSTACERIN® SFO, Clariant), Polyglyceryl-10 Laurate (DERMOFEEL® G10L, Dr. Straetmans), Polyglyceryl-10 Mono/Dioleate (CAPROL® PGE-860, Abitec Corporation), Polyglyceryl-4 Isostearate (ISOLAN® GI 34, Evonik), Polyglyceryl-5 Laurate (DERMOFEEL® GSL, Dr. Straetmans or IMWITOR® 660, Cremer Oleo), Polyglyceryl-3 Polyricinoleate (DERMOFEEL® PR, Dr. Straetmans), Polyglyceryl-4 Oleate (PROTACHEM 100, Protameen Chemicals), Polyglyceryl-3 Methylglucose Distearate (TECO® CARE 450, Evonik), Isostearyl Diglyceryl Succinate (IMWITOR® 780 K, Cremer Oleo), Polyglyceryl-3 Laurate (HYDRAMOL TGL, Lubrizol Advanced Materials), Polyglyceryl-3 Behenate (PELEMOL® 3G 22, Phoenix Chemicals), Polyglyceryl-3 Stearate (DERMOFEEL® PS, Dr. Straetmans), Sorbitan Laurate (Span 20, Croda, Inc.), Sorbitan Stearate (Span 60, Croda, Inc.), Sorbitan Tristearate (Span 65, Croda, Inc.), Sorbitan Oleate (Span 80, Croda, Inc.), Sorbitan Sesquioleate (Span 83) and Sorbitan Isostearate (Span 120, Croda, Inc.).

Particularly preferred water-in-oil emulsifiers include Polyglyceryl-2 Dipolyhydroxystearate (DEHYMULS PGPH, BASF), PEG-30 Dipolyhydroxystearate (DEHYMULS® LE, BASF), Glyceryl Stearate (CUTINA® GMS, BASF), Polyglyceryl-2 Diisostearate (DERMOL® DGDIS, Alzo International), Sorbitan Trioleate (SPAN 85, Croda, Inc.), Sorbitan Palmitate (Span 40, Croda, Inc.), Diisostearoyl Polyglyceryl-3 Dimer Dilinoleate (ISOLAN® PDI, Evonik), and Polyglyceryl-4 Diisostearate/Polyhydroxystearate/Sebacate (ISOLAN® GPS, Evonik). The total concentration of water-in-oil emulsifiers can range from about 0.05% to about 5.0% by total weight of the oil-in-water emulsion, or more preferably from about 0.05% to about 2.0% (by total weight of the oil-in-water emulsion), or from about 0.25% to about 1.0% (by total weight of the oil-in-water emulsion).

The importance of selecting appropriate emulsifiers for the skin protective formulation will be demonstrated throughout this disclosure. The following example of an emulsion in Table 1 is provided for initial comparative purposes. The pre-immersion wVTR of the emulsion in Table 1 was measured. The emulsion of Table 1 includes materials with an inherent wVTR less than 30 g/m²/hr less (Mineral Oil and Petrolatum) whose combined concentration did not exceed 15% (by weight of the emulsion), and included Water at a concentration exceeding 70%. However, this emulsion in Table 1 did not include a plurality of water-in-oil emulsifiers. The pre-immersion wVTR of this emulsion was 49.80±3.80 g/m²/hr. The pre-immersion wVTR of the emulsion without a plurality of water-in-oil emulsifiers is much higher than the wVTR of exemplary embodiments of the present disclosure, which will be discussed in further detail below. Thus, not having the plurality of water-in-oil emulsifiers provided an inferior hydrophobic coating imparted to the surface.

TABLE 1

| Trade Name | Wt (%) |
|---|---|
| Part A | |
| Water | 74.29 |
| CoSept M | 0.30 |
| CoSept P | 0.10 |
| Citric Acid | 0.42 |
| Sodium Citrate | 0.32 |
| Glycerin | 5.00 |
| Urea | 0.64 |
| Part B | |
| Mineral Oil | 6.19 |
| Snow White Petrolatum | 3.75 |
| Dimethicone | 1.00 |
| Sunflower Oil | 3.38 |
| BHT | 0.02 |
| Ceteth-20 | 1.50 |
| Cetearyl Alcohol | 3.00 |
| Hydrocortisone | 0.10 |

Film-Forming Polymers:

According to the Personal Care Products Council, film forming polymers are materials which produce a continuous film on skin, hair, or nails. These materials can be used in cosmetics for diverse purposes, e.g., in forming facial masks, make-up films, hair-holding products or nail polishes. For the present disclosure, film forming polymers can not only lower the wVTR of the oil-in-water emulsion, but can also increase the resistance of the film to wash off with water or light detergent solutions. This is an important attribute as many industrial settings expose employees to excessive levels of water which may lead to ineffective protection without water-proof characteristics. Suitable film-forming polymers include, but are not limited to, Acrylates/Vinyl Neodecanoate Crosspolymer (ACULYN 38, Dow Chemical), Stearyl/Octyldodecyl Citrate Crosspolymer (COSMOSURF® CE-150, Siltech LLC), Isobutylene/Ethylmaleimide/Hydroxyethylmaleimide Copolymer (AQUAFLEX FX64, Ashland, Inc.), Polyacrylate-15 (SYNTRAN® PC 5208, Interpolymer Corporation), Polyurethane-34 (BAYCUSAN® C1000, Bayer MaterialScience LLC), Latex based emulsions (HYCAR® T9202, Lubrizol Advanced Materials), Acrylates/Steareth-20 Methacrylate Crosspolymer (ACULYN 88, Dow Chemical), Styrene/Acrylates Copolymer (DERMACRYL® E, National Starch), Polyurethane-32 (BAYCUSAN® C1003, Bayer Material Science LLC), C8-22 Alkyl Acrylates/Methacrylic Acid Crosspolymer (INTELIMER® 8600, Air Products), Acrylates Copolymer (AVALURE AC 120, Lubrizol Advanced Materials), Polyacrylate-15 and Polyacrylate-17 (SYNTRAN® PC 5227, Interpolymer Corporation), Polyester-7 (LEXFILM® SUN, Inolex Inc.), Acrylates/Steareth-20 Methacrylate Copolymer (ACULYN 22, Dow Chemical), Acrylates/Beheneth-25 Methacrylate Copolymer (ACULYN 28, Dow Chemical), PEG-150/Decyl Alcohol/SMDI Copolymer (ACULYN 44, Dow Chemical), Dimethicone/Vinyl Dimethicone Crosspolymer (DIOWGEL 6413, Centerchem, Inc.), Acrylates/Bis-Hydroxypropyl Dimethicone Crosspolymer (Gransil ASP, Grant Industries, Inc.), Bis-Hydroxypropyl Dimethicone/SMDI Copolymer (Silmer® UR-5050, Siltech LLC), Bis-PPG-15 Dimethicone/IPDI Copolymer, Octadecene/MA Copolymer (Stantiv® OMA-2, Vertellus Performance Materials, Inc.), Methacryloyl Ethyl Betaine/Acrylates Copolymer (Diaformer® Z-301N, Clariant International Ltd,) (Polyderm PPI-SI-WI, Alzo International), VP/Eicosene Copolymer (Ganex® V-220, Ashland Inc.), Vinyl Dimethicone/Methicone Silsesquioxane Crosspolymer (KSP-100, Shin-Etsu Chemical Co.), and Butylated PVP (GANEX® P-904 LC, Ashland Inc.).

Particularly preferred film forming polymers include PPG-12/SMDI Copolymer (POLYOLPREPOLYMER 2, Barnet Products), IPDI/PEG-15 Cocamine Copolymer (POLYDERM PPI-CA-15, Alzo Corporation), Acrylates Copolymer (EPITEX 66, Dow Chemical Company), Polyester-10 (LEXFILM® SPRAY, Inolex Inc.), Acrylates/C12-22 Alkyl Methacrylate Copolymer ALLIANZ OPT, PEG-8/SMDI Copolymer (POLYOLPREPOLYMER 15, Barnet Products), Acrylates Copolymer (SYNTRAN® 5190, Interpolymer Corporation), IPDI/PEG-15 Cocamine Copolymer (POLYDERM PPI-SA-15D, Alzo Corporation), Hydrogenated Dimer Dilinoleyl/Dimethylcarbonate Copolymer (COSMEDIA® DC, BASF), Styrene/Acrylates/Ammonium Methacrylate Copolymer, (SYNTRAN 5760, Interpolymer Corporation), Trimethylpentanediol/Adipic Acid/Glycerin Crosspolymer (LEXOREZ 200, Inolex), and PVP (Luviskol® K90, BASF).

It is contemplated that any combination of the aforementioned film forming polymers could be used, as well as combinations of other suitable film forming polymers, as are known by one of ordinary skill in the art. In some embodiments, the concentration of film-forming polymers ranges from about 0.05% to about 5.0% (by total weight of the oil-in-water emulsion), or more preferably from about 0.5% to about 2.0% (by total weight of the oil-in-water emulsion), or even more preferably from about 1.0% to about 2.0% (by total weight of the oil-in-water emulsion).

The importance of including an efficacious film forming polymer within the skin protective formulation will be demonstrated throughout this disclosure. An oil-in-water emulsion of the formulation of Table 2 is provided for initial comparative purposes. The post-immersion wVTR was measured for this emulsion that included a plurality of water-in-oil emulsifiers with a material having an inherent wVTR less than 30 g/m²/hr at a concentration less than 15% (by weight of the oil-in-water emulsion) and Water at a concentration that exceeds 70%. However, the emulsion of the formulation of Table 2 did not include a film forming polymer. As a result, despite having a fairly low inherent wVTR (33 g/m²/hr), the post-immersion wVTR exceeded 50 g/m²/hr (56.25+/−3.11 g/m²/hr). As a result, the protection this emulsion could provide employees in a wet work environment is less than the present invention.

TABLE 2

| Ingredient | Wt (%) |
|---|---|
| Water | 78.00 |
| Mineral Oil | 6.00 |
| Isopropyl Palmitate | 3.00 |
| Cetearyl Glucoside (and) Cetearyl | |
| Alcohol | 3.00 |
| Polyglyceryl-2 Dipolyhydroxstearate | 2.00 |
| C12-15 Alkyl Benzoate | 1.50 |
| Stearic Acid | 1.50 |
| Petrolatum | 1.00 |
| Propylene Glycol | 1.00 |
| Phenoxyethanol | 0.70 |
| Bisabolol | 0.50 |
| PEG-30 Dipolyhydroxystearate | 0.50 |
| PEG-40 Stearate | 0.50 |
| Hamamelis Virginiana Water, Alcohol | 0.50 |
| Methylparaben | 0.16 |
| Butylparaben | 0.06 |
| Ethylparaben | 0.04 |
| Isobutylparaben | 0.02 |
| Propylparaben | 0.02 |

### Optional Ingredients

In some embodiments, the oil-in-water emulsion can include other ingredients in addition to the base components as described above. Other optional ingredient classes are as follows:

### Emollients:

In one embodiment, the emulsions can optionally include one or more emollients, which typically act to soften, soothe, and otherwise lubricate and/or moisturize the skin. Suitable emollients that can be incorporated into the compositions include oils such as Ethylhexyl Stearate (Cetiol® 868, BASF), Ethylhexyl Isostearate (Dub ISO, Stearinerie Dubois Fils), C12-15 Alkyl Ethylhexanoate (Hetester FAO, Bernel Chemical Company), C12-15 Alkyl Lactate (Ceraphyl® 41, Ashland Inc.), Caprylic/Capric Glycerides (IMWITOR® 742, Cremer Oleo), Caprylic/Capric Trigycerides (Crodamol GTCC, Croda, Inc.), Cetyl Esters (Crodamol SS, Croda, Inc.), Cetearyl Isononanoate (Cetiol® SN, BASF Corporation), Cetearyl Dimethicone (Botanisil® CD-16, Botanigenics, Inc.), Cetyl Dimethicone (Abil® Wax 9801, Evonik Industries AG), Coco-Caprylate (Cetiol® C5, BASF Corporation), C12-15 Pareth-3 Benzoate (Dermol® 25-3B, Alzo International), Diethyl Sebacate (Pelemol® DES, Phoenix Chemical, Inc.), Diisostearyl Fumarate (Schercemol DISF Ester, Lubrizol Advanced Materials, Inc.), Helianthus Annuus (Sunflower) Seed Oil (Florasun® 90, Floratech), Isodecyl Laurate (Isostearene, Vevy Europe SpA), Lauryl Lactate (Ceraphyl® 31, Ashland Inc.), Octyldodecanol (Isofol 20 Alcohol, Sasol Germany GmbH Hamburg) Olive Oil PEG-7 Esters (Olivem® 300, B&T S.r.l.) PEG-75 Meadowfoam Oil (Meadowsol® 75:75, Elementis Specialties), Perfluorononylethyl Carboxydecyl Lauryl Dimethicone (Pecosil® FST-412, Phoenix Chemical, Inc.), Polyglyceryl-2 Isostearate (Cithrol PG21IS, Croda Europe, Ltd.), Tridecyl Stearate (Hest TDS, Global Seven Inc.), alkyl dimethicones, alkyl methicones, alkyldimethicone copolyols, phenyl silicones, alkyl trimethylsilanes, dimethicone, dimethicone crosspolymers, cyclomethicone, Lanolin and its derivatives, fatty esters, glycerol esters and derivatives, propylene glycol esters and derivatives, alkoxylated carboxylic acids, alkoxylated alcohols, fatty alcohols, and combinations thereof.

The emulsions may include one or more emollients in a concentration of from about 0.01% to about 20.0% (by total weight of the emulsion), or from about 0.05% to about 10.0% (by total weight of the emulsion), or from about 0.10% to about 5.0% (by total weight of the emulsion).

### Preservatives:

The emulsions may include various preservatives to increase shelf life. Some suitable preservatives that may be used in the present disclosure include, but are not limited to Phenoxyethanol, Caprylyl Glycol, Chlorphenesin, 1,2-Hexanediol, Tropolone, Hydroxyacetophenone, Caprylhydroxamic Acid, Glyceryl Caprylate, Sorbic Acid, Gallic Acid, DMDM Hydantoin (e.g., GLYDANT, available from Lonza, Inc., Fair Lawn, N.J.); EDTA and salts thereof; Iodopropynyl Butylcarbamate; benzoic acid esters (parabens), such as Methyl paraben, Propyl paraben, Butyl paraben, Ethyl paraben, Isopropyl paraben, Isobutyl paraben, Benzyl paraben, Sodium Methyl paraben, and Sodium Propyl paraben; 2-Bromo-2-nitropropane-1,3-diol; Benzoic Acid; and the like. Other suitable preservatives include those sold by Sutton Labs Inc., Chatham, N.J., such as “GERMALL® 115” (Imidazolidinyl Urea), “GERMALL® II” (Diazolidinyl Urea), and “GERMALL® PLUS” (Diazolidinyl Urea and Iodopropynyl Butylcarbamate).

The concentration of the preservative in the emulsions is dependent on the relative concentrations of other components present within the emulsion. For example, in some embodiments, the preservative is present in the emulsions in a concentration between about 0.001% to about 5.0% (by total weight of the emulsion), in some embodiments between about 0.01% to about 3.0% (by total weight of the emulsion), and in some embodiments, between about 0.05% to about 1.0% (by total weight of the emulsion).

### Fragrance:

Another component that may be suitable for addition to the emulsions is a fragrance. Any compatible fragrance may be used. Typically, the fragrance is present in a concentration from about 0% to about 5.0% (by total weight of the emulsion), and more typically from about 0.01% to about 3.0% (by total weight of the emulsion). In one desirable embodiment, the fragrance will have a clean, fresh and/or neutral scent to create an appealing delivery vehicle for the end consumer.

### pH Adjusting Agent:

The emulsions of the present disclosure may further include a pH-adjusting agent. Such agents are desirable for the creation of emulsions having a pH at or near that of human skin. Therefore, the pH will typically be adjusted as necessary so that the emulsions of the present disclosure have a pH of from 4 to 7, or more desirably, from 4.5 to 6.5. The pH can be adjusted by adding one or more pH-adjusting agents in an amount effective to provide such pH values (“effective amount”). Agents that may be used to adjust the pH of the emulsions include organic and inorganic acids and bases.

Acid pH-adjusting agents include organic acids which are relatively non-irritating. Such acids include Malic Acid, Citric Acid, Acetic Acid, Propionic Acid, Oxalic Acid, Glycolic Acid, Malonic Acid, Lactic Acid, Succinic Acid, Tartaric Acid, Aspartic Acid, Maleic Acid, Glutaric Acid, Glutamic Acid, Gluconic Acid, Sorbic Acid, Benzoic Acid, Ascorbic Acid, Salicylic Acid and mixtures thereof. In one aspect of the present disclosure, a desirable pH-adjusting agent is Malic Acid.

Basic pH-adjusting agents include Aminomethyl Propanol (AMP-95 2-Amino-2-Methyl-1-Propanol, The Dow Chemical Company), Calcium Glycinate (OriStar CAGY, Orient Stars LLC), Calcium Hydroxide (Calcium Hydroxide, USP 802, Brenntag Specialties Inc.), Diethanolamine (Dealan, Rita Corporation), Ethanolamine (Mealan, Rita Corporation), Sodium Citrate (Trisodium Citrate Dihydrate, Jungbunzlauer, Inc.), Sodium Glycolate (Glycolic Acid, Sodium Salt, Chelest Corporation), Sodium Hydroxide (Unichem SOHYD, Universal Preserv-A-Chem, Inc.), Triethanolamine (Tealan 85%, Rita Corporation), Trisodium Phosphate (Trisodium Phosphate Dodecahydrate, Independent Chemical Corp.) and Zinc Glycinate (OriStar ZNGY, Orient Stars LLC).

The amount of the pH-adjusting agent that is employed depends on the equivalent weight of the pH-adjusting agent and the desired pH. Typically, the pH-adjusting agent is used in a concentration of from about 0.05% to about 0.5% (by total weight of the emulsion). Desirable concentrations of a pH-adjusting agent in emulsions of the present disclosure can range from about 0.1% to about 0.5% (by total weight of the emulsion), and typically about 0.2% to about 0.3% (by total weight of the emulsion).

### Chelating Agent:

The antiperspirant emulsions may contain one or more chelating agents. The chelating agent tends to bind metals (e.g., Calcium ions, Magnesium ions) that may be present in the emulsion so as to enhance the efficiency of the emulsifier and the antimicrobial agent. Thus, the chelating agent may be considered to provide a level of antimicrobial activity to function as a preservative. The chelating agent may be used in a concentration that is effective to bind the aforementioned metals (hereinafter alternatively referred to as an “effective amount”), typically a concentration ranging from about 0.01% to about 0.9% (by total weight of the emulsion). In some embodiments, the chelating agents can be used in a concentration from about 0.05% to about 0.2% (by total weight of the emulsion), or from about 0.05% to about 0.10% (by total weight of the emulsion). Chelating agents and their use in personal cleansing emulsions are well known in the art. Exemplary chelating agents include Disodium EDTA (Versene NA, The Dow Chemical Company), Trisodium EDTA (Universene Na3, Universal Preserv-A-Chem, Inc.), Tetrasodium EDTA (Versene 100, The Dow Chemical Company), Tetrasodium Glutamate Diacetate (Dissolvine® GL-47-S, AkzoNobel Global Personal Care), Caprylhydroxamic Acid (Inolex, Inc.) and Tetrasodium Iminodisuccinate (Iminodisuccinic Acid Sodium Salt, Lanxess Deutschland GmbH).

### Rheology Modifiers:

Various oil and water soluble rheology modifiers can be included within the formulation to increase formulation viscosity and stability of the emulsion. A non-exhaustive list of rheology modifiers includes Acrylates Copolymer (Rheocare® TTA, BASF Corporation), Acrylates/C10-30 Alkyl Acrylate Crosspolymer (Pemulen® TR-2, Lubrizol Advanced Materials, LLC), Carbomer (Carbopol® 980 Polymer, Lubrizol Advanced Materials, Inc.) Magnesium Aluminum Silicate (Veegum® Ultra, Vanderbilt Minerals, LLC), Quaternium-18 Hectorite (Lucentite SAN-P, Kobo Products, Inc.) Bentonite (Bentolite® WH, BYK Additives Inc), Hectorite (Bentone® EW, Elementis Specialties), Xanthan Gum (Rhodicare® XC, Rhodia Inc.), Gellan Gum (Kelcogel®, CP Kelco), Cellulose Gum (Aqualon Cellulose Gum, Ashland Inc.), Microcrystalline Cellulose (Avicel® PH-101, NF, FMC Corporation), Methylcellulose (Benecel Methylcellulose, Ashland Inc.), Hydroxypropylcellulose (Klucel Hydroxypropylcellulose (Ashland Inc.), Hydroxypropyl Methylcellulose (Methocel E50 P AMC HPMC, The Dow Chemical Company), Beeswax (Kahl GmbH & Co. KG), Ceresin Wax (Ceresine Wax White SP-101, Strahl & Pitsch, Inc.), Paraffin (Paraffin Wax 120/127, Rita Corporation), Cetyl Alcohol (Crodacol C-95, Croda, Inc.), Kaolin (2457 Kaolin USP, Brenntag Specialties Inc.), Zea Mays (Corn) Starch (Purity 21C, AkzoNobel Global Personal Care), Solanum Tuberosum (Potato) Starch (XyPure PT, Xytrus), Tapioca Starch (Tapioca Pure, AkzoNobel Global Personal Care), Carrageenan (Viscarin® GP109 NF, FMC Corporation) and combinations thereof.

When selecting rheology modifiers, it is important to ensure that their inclusion does not adversely impact the wVTR or contact angle values of the overall formulation. The formulations in Table 3 demonstrate that as the content of more hydrophilic rheology modifiers such as Xanthan Gum and Aluminum Magnesium Silicate increases, the pre-immersion wVTR values increase. Without being bound to any theory, it is believed that these macromolecules interfere with the uniformity of the hydrophobic coating, creating gaps which allow water vapor to escape.

TABLE 3

| Trade Name | INCI Name | Wt % | Wt % | Wt % |
|---|---|---|---|---|
| Part A | | | | |
| Water | Water | 73.25 | 71.45 | 70.45 |
| Glycerin | Glycerin | 1.00 | 1.00 | 1.00 |
| Ultrez 30 | Carbomer | 0.30 | 0.00 | 0.00 |
| Natrasol 250 HR | Hydroxyethylcellulose | 0.00 | 1.00 | 1.00 |
| Veegum Ultra | Aluminum Magnesium Silicate | 0.00 | 1.00 | 2.00 |
| Rhodicare XC | Xanthan Gum | 0.15 | 0.25 | 0.25 |
| Part B | | | | |
| Cetiol CC | Dicapryl Carbonate | 5.00 | 5.00 | 5.00 |
| Cutina GMS | Glyceryl Stearate | 3.00 | 3.00 | 3.00 |
| Span 40 | PEG-40 Stearate | 0.50 | 0.50 | 0.50 |
| Span 85 | Sorbitan Trioleate | 1.00 | 1.00 | 1.00 |
| Sonneconne CM | Petrolatum | 10.00 | 10.00 | 10.00 |
| Part C | | | | |
| Allianz OPT | Acrylates/C12-22 Alkyl Methacrylate | 1.75 | 1.75 | 1.75 |
| | Copolymer | | | |
| Dry Flo Elite BN | Aluminum Starch Octenylsuccinate (and) | 2.50 | 2.50 | 2.50 |
| | Boron Nitride | | | |
| Allantoin | Allantoin | 0.75 | 0.75 | 0.75 |
| Verstatil TBO | Triethyl Citrate (and) Caprylyl Glycol (and) | 0.80 | 0.80 | 0.80 |
| | Benzoic Acid | | | |
| Sodium Hydroxide (20%) | Sodium Hydroxide | QA | QA | QA |
| | Pre-Immersion WVTR (g/m²/hr) | 20.37 | 35.80 | 45.34 |
| | STDEV. | 4.21 | 5.29 | 1.37 |

### Skin Protectants:

The cosmetic oil-in-water emulsions of the present disclosure would allow delivery of ingredients recognized by the Food and Drug Administration as Skin Protectants under the appropriate Over the Counter Drug Monograph. In one embodiment, the skin protectants include Allantoin, Dimethicone or Zinc Oxide. Other suitable skin protectants may be included in the following amounts: Allantoin (0.5 to 2.0%), Aluminum hydroxide gel (0.15 to 5.0%), Calamine (1.0 to 25.0%), Cod liver oil (5.0 to 13.56%), Colloidal oatmeal (at least 0.007%), Dimethicone (1.0 to 29.85%), Glycerin (20.0 to 45.0%), Kaolin (4.0 to 20.0%), Lanolin (12.5 to 29.85%), Topical Starch (10.0 to 98.0%), Zinc Acetate (0.1 to 2.0%), Zinc Carbonate (0.2 to 2.0%), and Zinc Oxide (1.0 to 25.0%). All percentages are by total weight of the emulsion.

### Preparation of Emulsions

The oil-in-water emulsions of the present disclosure include a water phase and an oil phase which are prepared separately and then combined at high temperatures to form the emulsion. Generally, both the water phase and the oil phase are prepared by adding ingredients under constant agitation while heating to 75° C. One ordinarily skilled in the art is familiar with the appropriate methods and equipment necessary. For these emulsions, the use of an IKA mixer with a propeller mixing head at a speed necessary to ensure a vortex is achieved (500 rpm) was sufficient to ensure that the phases were uniformly mixed. Once both phases reach 75° C., the oil phase is added to the water phase under homogenization at 5000 rpm for 5 minutes using a Silverson Homogenizer. The emulsion is returned to propeller based mixing until it cools to 35° C. or lower. The pH of the formulation is then adjusted to the appropriate pH (usually 5.25) using a pH adjusting agent, such as Sodium Hydroxide and/or Malic Acid.

### EXAMPLES

### Example 1

A variety of commercial water-in-oil emulsifiers were added at 1% w/w to the base emulsion systems provided in Tables 4 and 5. Due to the number of emulsifiers and their oil soluble nature, stock solutions for the oil and water phases were created. The emulsifier of interest was added to the appropriate stock oil phase which was heated to 75° C. to ensure a homogenous phase was created. This complete oil phase was then added to an appropriate amount of the stock water phase, which was also heated to 75° C., and homogenized for 5 minutes at 5000 rpm. The sample was then cooled to room temperature at which point the pH was adjusted to approximately 5.25. As shown in Table 6, the following water-in-oil (“W/O”) emulsifiers lowered the wVTR of the base formulation: DEHYMULS PGPH, DEHYMULS LE, DERMOL DGDIS, ISOLAN PDI, and ISOLAN GPS.

TABLE 4
Stock Water Phase

| | | Wt % in | |
|---|---|---|---|
| | | Finished | Wt % in Stock |
| Part A | | Formulation | Solution |
| Water | Water | 75.15 | 97.60 |
| COSEPT M | Methylparaben | 0.20 | 0.26 |
| COSEPT P | Propylparaben | 0.10 | 0.13 |
| ELSTAB CPN | Chlorphenesin | 0.10 | 0.13 |
| ULTREZ 30 | Carbomer | 0.30 | 0.39 |
| Glycerin | Glycerin | 1.00 | 1.30 |
| TIC Prehydrated Xanthan | Xanthan Gum | 0.15 | 0.19 |

TABLE 5
Stock Oil Phase

| | | Wt % in | |
|---|---|---|---|
| Trade Name | | Finished | Wt % in Stock |
| Part B | INCI Name | Formulation | Solution |
| SONNECONNE CM | Petrolatum | 10.0 | 43.5 |
| CETIOL CC | Dicapryl Carbonate | 10.0 | 43.5 |
| BRIJ L4 | Laureth-4 | 2.0 | 9.0 |
| BRIJ S21 | Steareth-21 | 1.0 | 4.0 |

TABLE 6

| | | Pre-Immersion | |
|---|---|---|---|
| | | wVTR | |
| No. | W/O Co-Emulsifier | (g/m²/hr) | Standard Deviation |
| 1-1 | None | 30.98 | 12.37 |
| 1-2 | DEHYMULS PGPH | 9.87 | 3.09 |
| 1-3 | DEHYMULS LE | 13.41 | 0.44 |
| 1-4 | DERMOL DGDIS | 20.19 | 9.00 |
| 1-5 | ISOLAN PDI | 24.71 | 2.37 |
| 1-6 | ISOLAN GPS | 27.43 | 8.33 |
| 1-7 | SOFTISAN 378 | 33.99 | 8.42 |
| 1-8 | CREMERCOOR PG2 C10 | 34.17 | 0.92 |
| 1-9 | HOSTACERIN SFO | 34.78 | 14.59 |
| 1-10 | CREEMERCOOR PG4 | 37.29 | 5.33 |
| | COCOATE | | |
| 1-11 | DERMOFEEL G10L | 38.89 | 10.90 |
| 1-12 | CAPROL PGE-860 | 42.27 | 2.94 |
| 1-13 | ISOLAN GI 34 | 43.21 | 3.57 |
| 1-14 | DERMOSOFT DGMC | 43.60 | 0.41 |
| 1-15 | IMWITOR 600 LIQUID | 43.92 | 0.42 |
| 1-16 | DERMOFEEL G5L | 45.77 | 6.03 |
| 1-17 | DERMOFEEL PR | 46.29 | 4.61 |
| 1-18 | PROACHEM 100 | 46.32 | 0.64 |
| 1-19 | TEGOCARE PBS 6 | 47.02 | 1.17 |
| 1-20 | CREMOPHOR GS 32 | 47.09 | 1.45 |
| 1-21 | TEGOCARE 450 | 49.27 | 1.47 |
| 1-22 | IMWITOR 780 K | 53.27 | 13.84 |
| 1-23 | HYDRAMOL TGL | 53.59 | 7.56 |
| 1-24 | PELEMOL 3G 22 | 55.65 | 2.49 |
| 1-25 | DERMOFEEL PS | 55.90 | 15.03 |

### Example 2

A variety of commercial film forming polymers were added at 1.0% active polymer (by total weight of the emulsion) to the base emulsions system disclosed in Table 7. For oil-soluble film-forming polymers, two stock emulsions were created (see Tables 4 and 5). The film-forming polymers of interest were added to the stock oil phase which was previously heated to 75° C. to ensure that a homogenous phase was created. This phase was then added to an appropriate amount of the stock water phase also heated to 75° C. and homogenized for 5 minutes at 5000 rpm. The resulting sample was cooled to room temperature. Once at room temperature, the pH was adjusted to approximately 5.25. For water-soluble film-forming polymers, the base emulsion was completely formed following the same general procedure as above with the film-forming polymer being directly added to an aliquot of the formulation. As shown by the results in Table 8, the following film-forming polymers significantly lowered the wVTR value of the base formulation: POLYOLPREPOLYMER 2, POLYDERM PPI-CA-15, EPITEX 66, LEXFILM SPRAY, ALLIANZ OPT, POLYOLPREPOLYMER 15, SYNTRAN 5190, POLYDERM PPI-SA-15D, COSMEDIA DC, SYNTRAN 5760, LEXOREZ 200 and CELVOL 523.

TABLE 7

| TRADE NAME | INCI NAME | Wt % |
|---|---|---|
| Part A | | |
| Water | Water | 75.15 |
| COSEPT M | Methylparaben | 0.20 |
| COSEPT P | Propylparaben | 0.10 |
| ELSTAB CPN | Chlorphenesin | 0.10 |
| ULTREZ 30 | Carbomer | 0.30 |
| Glycerin | Glycerin | 1.00 |
| TIC Prehydrate Xanthan | Xanthan Gum | 0.15 |
| Part B | | |
| CETIOL CC | Dicapryl | 10.00 |
| | Carbonate | |
| SONNECONNE CM | Petrolatum | 10.00 |
| BRIJ L4 | Laureth-4 | 2.00 |
| BRIJ 721 | Steareth-21 | 1.00 |

TABLE 8

| | | Pre-Immersion | |
|---|---|---|---|
| | | wVTR | Standard |
| No. | Film Forming Polymer | (g/m²/hr) | Deviation |
| 2-1 | None | 30.98 | 12.37 |
| 2-2 | POLYOLPREPOLYMER 2 | 12.99 | 8.97 |
| 2-3 | POLYDERM PPI-CA-15 | 18.50 | 3.53 |
| 2-4 | EPITEX 66 | 19.37 | 10.64 |
| 2-5 | LEXFILM SPRAY | 21.82 | 8.84 |
| 2-6 | ALLIANZ OPT | 24.18 | 6.08 |
| 2-7 | POLYOLPREPOLYMER 15 | 24.40 | 9.38 |
| 2-8 | SYNTRAN 5190 | 25.26 | 9.72 |
| 2-9 | POLYDERM PPI-SA-15D | 25.31 | 8.15 |
| 2-10 | COSMEDIA DC | 25.42 | 4.30 |
| 2-11 | SYNTRAN 5760 | 25.85 | 4.96 |
| 2-12 | LEXOREZ 200 | 28.27 | 6.29 |
| 2-13 | PVP | 28.44 | 6.01 |
| 2-14 | ACULYN 38 | 30.86 | 5.69 |
| 2-15 | COSMOSURF CE-150 | 32.34 | 10.93 |
| 2-16 | AQUAFLEX FX64 | 32.53 | 15.73 |
| 2-17 | SYNTRAN 5208 | 33.50 | 7.59 |
| 2-18 | BATCUSAN C1000 | 34.04 | 4.78 |
| 2-19 | HYCAR T9202 | 34.11 | 1.47 |
| 2-20 | ACULYN 88 | 34.30 | 4.68 |
| 2-21 | DERMACRYL E | 36.09 | 2.94 |
| 2-22 | BATCUSAN C1003 | 36.55 | 1.71 |
| 2-23 | INTELIMER 8600 | 40.28 | 0.73 |
| 2-24 | AVALURE AC 120 | 41.09 | 10.49 |
| 2-25 | SYNTRAN PC 5227 | 43.65 | 2.57 |
| 2-26 | LEXFILM SUN | 44.53 | 5.99 |
| 2-27 | ACULYN 22 | 45.07 | 4.68 |
| 2-28 | ACULYN 28 | 45.26 | 2.00 |
| 2-29 | ACULYN 44 | 50.55 | 4.94 |
| 2-30 | DIOWGEL 6413 | 59.66 | 2.65 |
| 2-31 | GANEX P-904 LC | 63.62 | 8.04 |

### Example 3

Emulsifiers and film forming polymers that were found effective at lowering the wVTR (see Examples 1 and 2) were combined within a base formulation as described in Example 1 and 2. The various water-soluble film-forming polymers were added to the formulation after the emulsion formation. The wVTR of the resulting formulations (Table 9) were measured following water immersion for 40 minutes. As shown by Table 10, the following combinations of emulsifier and film-forming polymer maintained the wVTR value less than 40 g/m²/hr following water immersion: ISOLAN PDI with ALLIANZ OPT, EPITEX 66 or SYNTRAN 5760; and DERMOL DGDIS with SYNTRAN 5760 or SYNTRAN 5190.

TABLE 9

| | | Base Formula | | | |
|---|---|---|---|---|---|
| Trade Name | INCI Name | 3-1 | 3-2 | 3-3 | 3-4 |
| Part A | | | | | |
| Water | Water | 73.8 | 73.8 | 73.8 | 73.8 |
| COSEPT M | Methylparaben | 0.3 | 0.3 | 0.3 | 0.3 |
| ELSTAB CPN | Chlorphenesin | 0.1 | 0.1 | 0.1 | 0.1 |
| ULTREZ 30 | Carbomer | 0.5 | 0.5 | 0.5 | 0.5 |
| GLYCERIN | Glycerin | 1.0 | 1.0 | 1.0 | 1.0 |
| RHODICARE XC | Xanthan Gum | 0.3 | 0.3 | 0.3 | 0.3 |
| PART B | | | | | |
| CETIOL CC | Dicapryl Carbonate | 10.0 | 10.0 | 10.0 | 10.0 |
| SONNECONNE | Petrolatum | 10.0 | 10.0 | 10.0 | 10.0 |
| CM | | | | | |
| BRIJ 721 | Steareth-21 | 1.0 | 1.0 | 1.0 | 1.0 |
| BRIJ 4 | Laureth-4 | 2.0 | 2.0 | 2.0 | 2.0 |
| ISOLAN PDI | Diisostearoyl Polyglyceryl-3 | 1.0 | — | — | — |
| | Dimer Dilinoleate | | | | |
| DERMOL DGDIS | Polyglyceryl-2 Diisostearate | — | 1.0 | — | — |
| DEHYMULS PGPH | Polyglyceryl-2 | — | — | 1.0 | — |
| | Dipolyhydroxystearate | | | | |
| DEHYMULS LE | PEG-30 Dipolyhydroxystearate | — | — | — | 1.0 |

TABLE 10

| | | | Post-Immersion | |
|---|---|---|---|---|
| Base | | Wt % (As | wVTR | |
| Formulation | Film Forming Polymer | Supplied) | (g/m²/hr) | STDEV |
| 3-1 | ALLIANZ OPT | 2.22 | 34.85 | 4.38 |
| 3-1 | EPITEX 66 | 2.22 | 31.60 | 0.88 |
| 3-1 | SYNTRAN 5760 | 2.40 | 37.71 | 3.78 |
| 3-1 | SYNTRAN 5190 | 2.40 | 42.76 | 10.50 |
| 3-2 | ALLIANZ OPT | 2.22 | 41.16 | 10.00 |
| 3-2 | EPITEX 66 | 2.22 | 51.48 | 14.02 |
| 3-2 | SYNTRAN 5760 | 2.40 | 24.50 | 14.91 |
| 3-2 | SYNTRAN 5190 | 2.40 | 29.40 | 9.24 |
| 3-3 | ALLIANZ OPT | 2.22 | 45.84 | 1.03 |
| 3-3 | EPITEX 66 | 2.22 | 45.52 | 1.25 |
| 3-4 | ALLIANZ OPT | 2.22 | 54.67 | 12.94 |
| 3-4 | EPITEX 66 | 2.22 | 59.04 | 7.94 |

### Example 4 Film-Forming Polymers and Emulsifiers: Water Resistance Testing

The level of oil soluble ingredients was lowered so that the total concentration of oil phase in the composition was no more than 16.5% by total weight of the composition, see Table 11. Collagen films were coated with the resulting emulsions and immersed in water for 4 hours to simulate one-half of a typical work shift in a wet work environment. As shown in Table 12, the use of either the ISOLAN PDI or ISOLAN GPS in combination with ALLIANZ OPT PG Polymer was the most effective in maintaining a wVTR value below 30 g/m²/hr, whereas including none, or either an emulsifier or film forming polymer alone resulted in a wVTR value greater than 50 g/m²/hr.

TABLE 11

| Trade Name | INCI Name | Wt % |
|---|---|---|
| Part A | | |
| Water | Water | 82.65 |
| COSEPT M | Methylparaben | 0.30 |
| ELSTAB CPN | Chlorphenesin | 0.10 |
| ULTREZ 30 | Carbomer | 0.30 |
| GLYCERIN | Glycerin | 1.00 |
| RHODICARE XC | Xanthan Gum | 0.15 |
| PART B | | |
| CETIOL CC | Dicapryl | 5.00 |
| | Carbonate | |
| SONNECONNE CM | Petrolatum | 7.50 |
| BRIJ 721 | Steareth-21 | 1.00 |
| BRIJ 4 | Laureth-4 | 2.00 |

TABLE 12

| | | | | | Post-Immersion | |
|---|---|---|---|---|---|---|
| | W/O Emulsifier | | Film Forming Polymer | | wVTR | |
| | Trade Name | Wt % | Trade Name | Wt % | (g/m²/hr) | Std Dev |
| 4-1 | None | 0.00 | None | 0.00 | 51.05 | 7.83 |
| 4-2 | ISOLAN PDI | 0.25 | None | 0.00 | 55.09 | 10.15 |
| 4-3 | ISOLAN PDI | 1.00 | None | 0.00 | 58.21 | 0.92 |
| 4-4 | None | 0.00 | ALLIANZ OPT PG POLYMER | 0.50 | 26.56 | 12.10 |
| 4-5 | None | 0.00 | ALLIANZ OPT PG POLYMER | 1.00 | 36.21 | 3.46 |
| 4-6 | None | 0.00 | ALLIANZ OPT PG POLYMER | 1.50 | 24.02 | 5.60 |
| 4-7 | None | 0.00 | ALLIANZ OPT PG POLYMER | 1.75 | 23.10 | 1.40 |
| 4-8 | ISOLAN PDI | 0.25 | ALLIANZ OPT PG POLYMER | 0.50 | 23.67 | 3.54 |
| 4-9 | ISOLAN PDI | 0.25 | ALLIANZ OPT PG POLYMER | 1.00 | 17.67 | 11.70 |
| 4-10 | ISOLAN PDI | 0.25 | ALLIANZ OPT PG POLYMER | 1.75 | 13.97 | 3.49 |
| 4-11 | ISOLAN GPS | 0.25 | None | 0.00 | 36.96 | 7.86 |
| 4-12 | ISOLAN GPS | 1.00 | None | 0.00 | 46.08 | 13.91 |
| 4-13 | ISOLAN GPS | 0.25 | ALLIANZ OPT PG POLYMER | 0.50 | 25.87 | 18.65 |
| 4-14 | ISOLAN GPS | 0.25 | ALLIANZ OPT PG POLYMER | 1.00 | 20.27 | 3.92 |
| 4-15 | ISOLAN GPS | 0.25 | ALLIANZ OPT PG POLYMER | 1.75 | 16.98 | 2.50 |

### Example 5

Another aspect of the present disclosure is depositing a film onto the skin that is highly breathable, while imparting an occlusive, hydrophobic film that resists wash-off. The two emulsions disclosed in Table 13 had a significantly higher oxygen permeability rate when compared to the following commercially available formulations: STOKO Lotion and generic Petrolatum, see Table 14. This indicates a substantially greater degree of breathability in comparison to these commercial products.

TABLE 13

| Trade Name | INCI Name | 5-1 | 5-2 |
|---|---|---|---|
| Part A | | | |
| Water | Water | 66.55 | 67.05 |
| COSEPT M | Methylparaben | 0.20 | 0.20 |
| COSEPT P | Propylparaben | 0.10 | 0.10 |
| ELSTAB CPN | Chlorphenesin | 0.10 | 0.10 |
| ULTREZ 30 | Carbomer | 0.50 | 0.50 |
| Glycerin | Glycerin | 1.00 | 1.00 |
| TIC Prehydrate | Xanthan Gum | 0.30 | 0.30 |
| Xanthan | | | |
| Part B | | | |
| SILKFLO 362 | Hydrogenated Polydecene | 10.00 | 10.00 |
| SONNECONNE CM | Petrolatum | 15.00 | 15.00 |
| BRIJ L4 | Laureth-4 | 2.00 | 2.00 |
| BRIJ 721 | Steareth-21 | 1.00 | 1.00 |
| ISOLAN PDI | Diisostearoyl Polyglyceryl-3 Dimer | 1.00 | 1.00 |
| | Dilinoleate | | |
| COSMOSURF CE | Stearyl/Octyldodecyl Citrate | 1.00 | 1.00 |
| 150 | Crosspolymer | | |
| Part C | | | |
| ALLANTOIN | Allantoin | 0.00 | 0.75 |
| Z-COAT | Zinc Oxide | 1.25 | 0.00 |

TABLE 14
Oxygen Permeability (cc/100 in² * day)

| | |
|---|---|
| STOKO Lotion | 0.0418 |
| STOKO Lotion | 0.0467 |
| Petrolatum | 0.4740 |
| Petrolatum | 0.6480 |
| Formulation 5-1 | 1.4900 |
| Formulation 5-1 | 1.6200 |
| Formulation 5-2 | 2.6700 |
| Formulation 5-2 | 3.0700 |

### Example 6

Within another example, the Formulation 5-2 from Table 13 was evaluated for the ability to prevent permeation of Sodium Lauryl Sulfate, a known irritant, through human cadaver skin. In addition to the formulation of this invention, the commercially available STOKO Lotion was also tested. As shown in Table 15, Formulation 5-2 prevented less Sodium Lauryl Sulfate to permeate through the cadaver skin as compared to the STOKO Lotion.

TABLE 15

| | SLS (ug/ml) | Std. Dev. |
|---|---|---|
| Formulation 5-2 | 0.17 | 0.14 |
| STOKO Lotion | 97.63 | 20.88 |

### Example 7

For many applications, a formulation with a high contact angle of water may also have utility in providing a protective coating to the skin. To impart a greater water contact angle to the formulation, it was found necessary to change the emulsifiers used. Within Table 16, the impact that various emulsifiers, emollients and rheology modifiers have on the contact angle is shown. Overall, selection of the emulsifier was the most critical factor in creating an emulsion with a high wVTR value. Generally, increasing the level of low HLB emulsifiers (water-in-oil emulsifiers), such as Glyceryl Stearate and Steareth-2, relative to more hydrophilic emulsifiers (oil-in-water emulsifiers) such as Steareth-20, PEG-100 Stearate and PEG-40 Stearate, resulted in a higher contact angle.

TABLE 16

| | | 7-1 | 7-2 | 7-3 | 7-4 | 7-5 | 7-6 |
|---|---|---|---|---|---|---|---|
| TRADE NAME | INCI Name | Wt % | Wt % | Wt % | Wt % | Wt % | Wt % |
| Part A | | | | | | | |
| Water | Water | 71.20 | 67.70 | 67.70 | 67.70 | 70.70 | 70.70 |
| Ultrez 30 | Carbomer | 0.50 | 0.50 | 0.50 | 0.50 | 0.50 | 0.50 |
| Glycerin | Glycerin | 1.00 | 1.00 | 1.00 | 1.00 | 1.00 | 1.00 |
| Rhodicare XC | Xanthan Gum | 0.25 | 0.25 | 0.25 | 0.25 | 0.25 | 0.25 |
| Part B | | | | | | | |
| Cetiol CC | Dicapryl Carbonate | 5.00 | 5.00 | 5.00 | 5.00 | 5.00 | 5.00 |
| Lanolin | Lanolin | 3.00 | 3.50 | 3.50 | 3.50 | 3.50 | 3.50 |
| Brij 721 | Steareth-21 | 1.00 | 1.00 | 1.00 | 1.00 | — | — |
| Brij 4 | Laureth-4 | 2.00 | 2.00 | 2.00 | 2.00 | — | — |
| Cutina GMS | Glyceryl Stearate | — | — | — | — | 2.00 | 2.00 |
| Myrj 40 | PEG-40 Stearate | — | — | — | — | 1.00 | — |
| Mryj 100 | PEG-100 Stearate | — | — | — | — | — | 1.00 |
| Isolan PDI | Diisostearoyl Polyglyceryl- | 0.25 | 0.25 | 0.25 | 0.25 | 0.25 | 0.25 |
| | 3 Dimer Dilinoleate | | | | | | |
| Sonneconne CM | Petrolatum | 10.00 | 10.00 | 10.00 | 10.00 | 10.00 | 10.00 |
| NF WHITE | Beeswax | — | 3.00 | — | — | — | — |
| BEESWAX | | | | | | | |
| White Ceresine Wax | Ceresin Wax | — | — | 3.00 | — | — | — |
| SP-101 | | | | | | | |
| MICROCRYSTALLINE | Microcrystalline Wax | — | — | — | 3.00 | — | — |
| WAX, SP-18 | | | | | | | |
| Part C | | | | | | | |
| Allianz OPT | Acrylates/C12-22 | 1.75 | 1.75 | 1.75 | 1.75 | 1.75 | 1.75 |
| | Alkylmethacrylate | | | | | | |
| | Copolymer | | | | | | |
| Dry Flo Elite BN | Aluminum Starch | 2.50 | 2.50 | 2.50 | 2.50 | 2.50 | 2.50 |
| | Octenylsuccinate (and) | | | | | | |
| | Boron Nitride | | | | | | |
| Allantoin | Allantoin | 0.75 | 0.75 | 0.75 | 0.75 | 0.75 | 0.75 |
| Verstatil TBO | Triethyl Citrate (and) | 0.80 | 0.80 | 0.80 | 0.80 | 0.80 | 0.80 |
| | Caprylyl Glycol (and) | | | | | | |
| | Benzoic Acid | | | | | | |
| Sodium Hydroxide | Sodium Hydroxide | Q.S. | Q.S. | Q.S. | Q.S. | Q.S. | Q.S. |
| (20%) | | | | | | | |
| | Contact Angle | 7.2 | 21.0 | 18.1 | 16.9 | 50.7 | 54.9 |
| | Std. Dev. | 0.7 | 1.3 | 1.1 | 2.3 | 1.3 | 1.5 |

As shown in Table 17, several emulsifiers added at 1% gave a contact angle near 60° and a pre-immersion wVTR value between 30 and 40 g/m²/hr.

TABLE 17

| | | 7-7 | 7-8 | 7-9 | 7-10 | 7-11 | 7-12 | 7-13 |
|---|---|---|---|---|---|---|---|---|
| Trade Name | INCI Name | Wt % | Wt % | Wt % | Wt % | Wt % | Wt % | Wt % |
| Part A | | | | | | | | |
| Water | Water | 72.95 | 72.95 | 72.95 | 72.95 | 72.95 | 72.95 | 72.95 |
| Ultrez 30 | Carbomer | 0.50 | 0.50 | 0.50 | 0.50 | 0.50 | 0.50 | 0.50 |
| Glycerin | Glycerin | 1.00 | 1.00 | 1.00 | 1.00 | 1.00 | 1.00 | 1.00 |
| Rhodicare XC | Xanthan Gum | 0.25 | 0.25 | 0.25 | 0.25 | 0.25 | 0.25 | 0.25 |
| Part B | | | | | | | | |
| Cetiol CC | Dicapryl Carbonate | 5.00 | 5.00 | 5.00 | 5.00 | 5.00 | 5.00 | 5.00 |
| Cutina GMS | Glyceryl Stearate | 3.00 | 3.00 | 3.00 | 3.00 | 3.00 | 3.00 | 3.00 |
| Span 40 | PEG-40 Stearate | 0.50 | 0.50 | 0.50 | 0.50 | 0.50 | 0.50 | 0.50 |
| Isolan PDI | Diisostearoyl | 1.00 | | | | | | |
| | Polyglyceryl-3 Dimer | | | | | | | |
| | Dilinoleate | | | | | | | |
| Isolan GPS | Polyglyceryl-4 | | 1.00 | | | | | |
| | Diisostearate/ | | | | | | | |
| | Polyhydroxystearate/ | | | | | | | |
| | Sebacate | | | | | | | |
| Inwitor 600 | Polyglyceryl-3 | | | 1.00 | | | | |
| | Polyricinoleate | | | | | | | |
| Dehmuyls | Polyglyceryl-2 | | | | 1.00 | | | |
| PGPH | Dipolyhydroxystearate | | | | | | | |
| Cithrol | Polyglyceryl-2 | | | | | 1.00 | | |
| PG23IS | Triisostearate | | | | | | | |
| Span 85 | Sorbitan Trioleate | | | | | | 1.00 | |
| Span 65 | Sorbitan Tristearate | | | | | | | 1.00 |
| Sonneconne | Petrolatum | 10.00 | 10.00 | 10.00 | 10.00 | 10.00 | 10.00 | 10.00 |
| CM | | | | | | | | |
| Part C | | | | | | | | |
| Allianz OPT | Acrylates/C12-22 | 1.75 | 1.75 | 1.75 | 1.75 | 1.75 | 1.75 | 1.75 |
| | Alkylmethacrylate | | | | | | | |
| | Copolyme | | | | | | | |
| Dry Flo Elite | Aluminum Starch | 2.50 | 2.50 | 2.50 | 2.50 | 2.50 | 2.50 | 2.50 |
| BN | Octenylsuccinate | | | | | | | |
| | (and) Boron Nitride | | | | | | | |
| Allantoin | Allantoin | 0.75 | 0.75 | 0.75 | 0.75 | 0.75 | 0.75 | 0.75 |
| Verstatil TBO | Triethyl Citrate (and) | 0.80 | 0.80 | 0.80 | 0.80 | 0.80 | 0.80 | 0.80 |
| | Caprylyl Glycol | | | | | | | |
| | (and) Benzoic Acid | | | | | | | |
| Sodium | Sodium Hydroxide | Q.S. | Q.S. | Q.S. | Q.S. | Q.S. | Q.S. | Q.S. |
| Hydroxide | | | | | | | | |
| (20%) | | | | | | | | |
| | Contact Angle | 56.9 | 56.1 | 60.1 | 61.5 | 63.9 | 64.3 | 64.6 |
| | Std. Dev. | 1.7 | 0.8 | 1.8 | 2.0 | 2.4 | 2.0 | 1.2 |
| | Pre-Immersion | 28.5 | 40.8 | 30.6 | 38.3 | 41.6 | 36.7 | 39.9 |
| | wVTR | | | | | | | |
| | Std. Dev. | 12.1 | 2.8 | 7.6 | 5.7 | 4.2 | 10.7 | 5.1 |

### Test Methods

Water Vapor Transport Rate (wVTR)—Pre-Immersion

Water Vapor Transport Rate (wVTR) measures the occlusivity of a formulation using Fischer/Payne permeability cups (Fischer Scientific Cat. #13-338). The cups consist of a stainless steel cup and washer, a gasket, and 3 stainless steel clamps. A circle with radius of 1.75 cm is marked with a permanent marker on the center of a circular piece of collagen film (Butcher & Packer Supply Company). The collagen films are weighed and recorded. Based on the area, 19 mg of the target formulation were added to the collagen film, giving a total dosing of about 2 mg/cm². A gloved finger was then used to rub the formulation into a circular area drawn on the collagen film. The treated films are then equilibrated overnight (minimum of 12 hours) in 25° C./75 R.H. Just prior to the evaluation, the permeation cups are filled with 10 mL of deionized water. Each collagen film is set on the permeation cup with the formulation coated side facing up. A gasket and a washer are then placed on top of the collagen film to hold it in place. Three clamps are affixed to the Payne Permeability Cup to keep the assembly intact, allowing an exposed collagen film surface of approximately 10 cm² to be exposed to the atmosphere. The entire assemblage is then weighed and the results recorded. Each permeability cup is set up in an identical fashion until all samples of interest were applied with each treatment being run in triplicate. The weights of the permeability cups assembled were measured periodically and the results recorded. This data was used to determine the rate of water vapor transport through both treated and untreated films. The slope of the line from a linear regression of water loss as a function of time provides the rate of water loss through the formulation treated collagen film. The water vapor transport rate is then expressed as the rate per unit area is expressed as g/m²/hr. A lower wVTR indicates a formulation that is more occlusive and thus, has greater barrier attributes towards water. In all individual experiments, films coated with about 2 mg/cm² Petrolatum and duplicate samples of untreated films were performed to increase the overall power of statistical rankings and ensure the results for an individual experiment were reliable.

Water Vapor Transfer Rate (wVTR): Post Immersion

The water resistance of the skin protective formulation were determined by immersing individual coated collagen films in 40° C. water for a set duration, usually 40 minutes. Each specimen was placed into 100 g of water with the entire water bath being placed in a 40° C. oven to ensure the temperature did not change throughout the immersion period. Following immersion, samples were removed from the jars of water and spread onto the top of a Payne Permeability Cup. Samples were then placed back into the 40° C. oven for 1 hour or until the film was found dry. The dried films were then allowed to equilibrate in TAPPI conditions overnight prior to re-measuring wVTR.

### Solids Content

The solids content was obtained by placing approximately 1 gram of the formulation in a pre-weighed 2 ounce jar and, after 24 hours at 50° C., the percentage change in weight was calculated. Although other volatile components (fragrances, preservatives and some emollients) may suppress this value, the values presented provide a fairly representative indication of solids content. Special consideration is made for products where this approximation may not be accurate (e.g., emulsions with a high content of volatile silicones).

### Oxygen Permeability

All oxygen permeability testing was performed by MOCON using the OXTRAN 2/21 Oxygen Permeability Instrument using test standard ASTM F 1927. The OXTRAN incorporates two diffusion cells. Each diffusion cell has two halves, a test gas (oxygen) side and a carrier gas (nitrogen) side. The cells are configured in a vertical arrangement, where the cell covers (test gas side of the diffusion cell) are secured in place with a yoke and clamp system. During the test, oxygen test gas flows on one side of the film and a nitrogen carrier gas sweeps across the other side of the film. Oxygen will permeate across the film from the high concentration side to the low concentration side. The permeating gas (flux) is swept by the nitrogen carrier gas stream to the OXTRAN's COULOX detector, where it is quantified. A corresponding transmission rate is calculated. A normal test within our lab will run for up to 100 hours or until equilibrium is reached (whichever occurs first). MOCON defines equilibrium as a point in which the transmission rate becomes stable (≦1% change in oxygen transmission rate or for low transmitters±the noise level of the instrument) over 24 hours. Since both the oxygen test gas and the nitrogen carrier gas are at equal pressures (ambient pressure), this testing methodology is also known as an isostatic test method. Additionally, barometric pressures variations due to location and weather fronts are automatically compensated within the instrument. The following experimental conditions were used for oxygen permeability testing: Test Humidity of 50%, Test Gas Pressure of 760 mm Hg, Test Temperature of 25° C.

### Sodium Lauryl Sulfate (SLS) Permeation

A dosage of 2 mg/cm² was applied to intact human epidermis in triplicate and allowed to dry overnight. The specimens were then placed into a Franz Cell permeation apparatus available from PERMEGEAR (#4G-01-00-11.28-08) with a 11.28 mm diameter and 8 mL receptor port. A total of 0.5 ml of 1% SLS within Phosphate buffered saline (Aldrich Cat #: P3818-10PK) pH 7.4, was applied to the solution compartment. The receptor solution also consisted of Phosphate buffered saline with sampling taking place at 4, 8, 24, 30, and 48 hours.

The concentration of Sodium Lauryl Sulfate was analyzed using High Performance Liquid Chromatography (HPLC). For HPLC measurements, an SLS stock standard solution was prepared in the following manner: Approximately 100 mg of SLS standard (Sigma L-4509 99% purity) was accurately weighed into a 20 ml scintillation vial. Milli-Q water was added until the total weight was 10 gms in the vial. The total weight was recorded to prepare this stock standard. Aliquots were taken from this standard to prepare the calibration standards. Nineteen aliquots were taken from the SLS working standard solution and transferred to nineteen separate 20 ml scintillation vials. Milli-Q water was added to each vial for a total of 10 grams in each vial. Only nine of the standard concentrations were plotted to generate a calibration standards. The concentrations ranged from 0.005 μg/ml to 3.300 Ng/ml. Ammonium Acetate was used as the reagent for analysis and prepared in the following manner: 100 mM Ammonium Acetate (pH 5.4) Solution—To a 1 L volumetric flask containing approximately 800 ml of Milli-Q water, add 7.71 gm of ammonium acetate (OPTIMA LC/MS—Fisher Scientific A114-50) and sonicate to dissolve. Dilute to volume with water. Adjust pH to 5.4 with concentrated glacial acetic acid (EMD AX0074-6) and filter through a 0.2 μm 47 mm nylon filter membrane (Pall Life Science P/N 66602).

All analysis was performed using a AGILENT 1290 Infinity Binary System with 1290 INFINITY Autosampler with a SIELC 5 μm Primesep B2 Column (2.1×100 mm) Part # B2-21.100.0510 and PRIMESEP B2 guard column Part #B2-21.G.0510 both commercially available through SIELC Technologies. The column temperature was kept at 40° C., the flow rate at 0.5 ml/min and injection volume of 2 μl. The following reagent gradient for the 100 mM Ammonium Acetate pH 5.4 (A) and Acetonitrile (B); 0 minutes: 60% A/40% B, 3.0 minutes: 20% A/80% B, 3.1 minutes: 60% A/40% B.

### Contact Angle

A dosage of 2 mg/cm² of the formulation was applied to VITRO-SKIN® N-19 (IMS, Inc.) in a uniform manner with a gloved finger prior to equilibrating overnight at ambient temperature and humidity. Contact angle measurements were then determined using the DSA 100 (KRUSS GmbH) drop shape analyzer. A total of 30 μl of deionized water (18.2Ω) was added to the sample with a total of 280-320 measurements being used to determine the average contact angle over an approximate 30 second time period. Three specimens per sample were then used to calculate the average contact angle and the standard deviation for the treatment under study. The contact angle of the untreated VITRO-SKIN was approximately 60 degrees.

### EMBODIMENTS

### Embodiment 1

A cosmetic oil-in-water emulsion comprising: water at a concentration of at least about 70% by total weight of the cosmetic oil-in-water emulsion; a plurality of water-in-oil emulsifiers; a base oil; the base oil having a wVTR of about 1.0 to 30 g/m²/hr and being at a concentration of no more than about 15% by weight of the cosmetic oil-in water emulsion; an oil-in-water emulsifier; and a film-forming polymer.

### Embodiment 2

The cosmetic oil-in-water emulsion of embodiment 1, wherein the cosmetic oil-in-water emulsion has a post-immersion wVTR value of between about 1.0 to about 40 g/m²/hr and an Oxygen Permeability of at least about 1.0 cc/100 in² per day.

### Embodiment 3

The cosmetic oil-in-water emulsion of any one of the preceding embodiments, wherein the base oil is selected from the group consisting of: Petrolatum, Mineral Oil, Hydrogenated Didecene, Hydrogenated Polydecene, Lanolin, and combinations thereof.

### Embodiment 4

The cosmetic oil-in-water emulsion of any one of the preceding embodiments, wherein the cosmetic oil-in-water emulsion has a pre-immersion wVTR value of less than about 30 g/m²/hr.

### Embodiment 5

The cosmetic oil-in-water emulsion of any one of the preceding embodiments, wherein the cosmetic oil-in-water emulsion has a contact angle greater than about 60°.

### Embodiment 6

The cosmetic oil-in-water emulsion of any of the preceding embodiments, wherein the plurality of water-in-oil emulsifiers provide a total concentration of about 0.05% to about 5.0% by total weight of the cosmetic oil-in-water emulsion.

### Embodiment 7

The cosmetic oil-in-water emulsion of any one of embodiments 1-5, wherein the plurality of water-in-oil emulsifiers provide a total concentration of about 0.05% to about 2.0% by total weight of the cosmetic oil-in-water emulsion.

### Embodiment 8

The cosmetic oil-in-water emulsion of any one of embodiments 1-5, wherein the plurality of water-in-oil emulsifier provide a total concentration of about 0.25% to about 1.0% by total weight of the cosmetic oil-in-water emulsion.

### Embodiment 9

The cosmetic oil-in-water emulsion of any one of the preceding embodiments, wherein the plurality of water-in-oil emulsifiers are selected from the group consisting of: Diisostearoyl Polyglyceryl-3 Dimer Dilinoleate, Polyglyceryl-4 Diisostearate/Polyhydroxystearate/Sebacate, Polyglyceryl-2 Diisostearate, Polyglyceryl-2 Dipolyhydroxystearate, PEG-30 Dipolyhydroxystearate, Glyceryl Stearate, and combinations thereof.

### Embodiment 10

The cosmetic oil-in-water emulsion of any one of the preceding embodiments, wherein the film-forming polymer is selected from the group consisting of Acrylates/C12-22 Alkyl Methacrylate Copolymer, Acrylates Copolymer, and combinations thereof; and wherein the film-forming polymer is at a concentration of about 0.05% to about 5.0% by total weight of the cosmetic oil-in-water emulsion.

### Embodiment 11

The cosmetic oil-in-water emulsion of any one of the preceding embodiments, wherein the film-forming polymer is at a concentration of about 0.5% to about 2.0% by total weight of the cosmetic oil-in-water emulsion.

### Embodiment 12

The cosmetic oil-in-water emulsion of any one embodiments 1-10, wherein the film-forming polymer is at a concentration of about 1.0% to about 2.0% by weight of the cosmetic oil-in-water emulsion.

### Embodiment 13

The cosmetic oil-in-water emulsion of any one of the preceding embodiments, wherein the oil-in water-emulsifier is selected from the group consisting of: Laureth-4, Steareth-21, PEG-100 Stearate, PEG-40 Stearate, and combinations thereof.

### Embodiment 14

The cosmetic oil-in-water emulsion of any one of the preceding embodiments, wherein the oil-in water-emulsifier is at concentration of about 0.1% to about 10.0% by total weight of the cosmetic oil-in-water emulsion.

### Embodiment 15

The cosmetic oil-in-water emulsion of any one of embodiments 1-13, wherein the oil-in water-emulsifier is at a concentration of about 1.0% to about 5.0% by total weight of the cosmetic oil-in-water emulsion.

### Embodiment 16

The cosmetic oil-in-water emulsion of any one of embodiments 1-13, wherein the oil-in water-emulsifier is at concentration of about 1.0% to about 3.0% by total weight of the cosmetic oil-in-water emulsion.

### Embodiment 17

The cosmetic oil-in-water emulsion of anyone of the preceding embodiments, further comprising a skin protectant, the skin protectant being selected from the group consisting of: Allantoin, Dimethicone, and Zinc Oxide.

### Embodiment 18

A cosmetic oil-in-water emulsion comprising: water at a concentration of about 70% to about 95% by total weight of the cosmetic oil-in-water emulsion; a base oil; an oil-in-water emulsifier; at least two water-in-oil emulsifiers that are selected from the group consisting of: Diisostearoyl Polyglyceryl-3 Dimer Dilinoleate, Polyglyceryl-4 Diisostearate/Polyhydroxystearate/Sebacate, Polyglyceryl-2 Diisostearate, Polyglyceryl-2 Dipolyhydroxystearate, PEG-30 Dipolyhydroxystearate, Glyceryl Stearate, and combinations thereof; and a film forming polymer.

### Embodiment 19

The cosmetic oil-in-water emulsion of embodiment 18, wherein the film forming polymer is selected from the group consisting of: Acrylates/C12-22 Alkyl Methacrylate Copolymer, Acrylates Copolymer, and combinations thereof, and wherein the film forming polymer is at a concentration of about 0.05% to about 5.0% by total weight of the cosmetic oil-in-water emulsion.

### Embodiment 20

The cosmetic oil-in-water emulsion of embodiment 18 or embodiment 19, wherein the oil-in water-emulsifier is selected from the group consisting of: Laureth-4, Steareth-21, PEG-100 Stearate, PEG-40 Stearate, and combinations thereof.

### Embodiment 21

The cosmetic oil-in-water emulsion of any one of embodiments 18-20, wherein the base oil has a wVTR of about 5 to 30 g/m²/hr and is at a concentration of no more than about 15% by total weight of the cosmetic oil-in-water emulsion.

### Embodiment 22

The cosmetic oil-in-water emulsion of any one of embodiments 18-21, wherein the cosmetic oil-in-water emulsion has a post-immersion wVTR value of between 0 to about 40 g/m²/hr and an Oxygen Permeability of at least about 1.0 (cc/100 in² per day).

### Embodiment 23

The emulsion of any one of embodiments 18-22, wherein the cosmetic oil-in-water emulsion has a pre-immersion wVTR value of less than 30 g/m²/hr.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

1. A cosmetic oil-in-water emulsion comprising:
water at a concentration of at least about 70% by total weight of the cosmetic oil-in-water emulsion;
a plurality of water-in-oil emulsifiers;
a base oil; the base oil having a wVTR of about 1.0 to 30 g/m²/hr and being at a concentration of no more than about 15% by total weight of the cosmetic oil-in water emulsion;
an oil-in-water emulsifier; and
a film-forming polymer.

2. The cosmetic oil-in-water emulsion of claim 1, wherein the cosmetic oil-in-water emulsion has a post-immersion wVTR value of between about 1.0 to about 40 g/m²/hr and an Oxygen Permeability of at least about 1.0 cc/100 in² per day.

3. The cosmetic oil-in-water emulsion of claim 1, wherein the base oil is selected from the group consisting of: Petrolatum, Mineral Oil, Hydrogenated Didecene, Hydrogenated Polydecene, Lanolin, and combinations thereof.

4. The cosmetic oil-in-water emulsion of claim 1, wherein the cosmetic oil-in-water emulsion has a pre-immersion wVTR value of less than about 30 g/m²/hr.

5. The cosmetic oil-in-water emulsion of claim 1, wherein the cosmetic oil-in-water emulsion has a contact angle greater than about 60°.

6. The cosmetic oil-in-water emulsion of claim 1, wherein the plurality of water-in-oil emulsifiers provide a total concentration of about 0.05% to about 5.0% by total weight of the cosmetic oil-in-water emulsion.

7. The cosmetic oil-in-water emulsion of claim 1, wherein the plurality of water-in-oil emulsifiers provide a total concentration of about 0.05% to about 2.0% by total weight of the cosmetic oil-in-water emulsion.

8. The cosmetic oil-in-water emulsion of claim 1, wherein the plurality of water-in-oil emulsifiers are selected from the group consisting of: Diisostearoyl Polyglyceryl-3 Dimer Dilinoleate, Polyglyceryl-4 Diisostearate/Polyhydroxystearate/Sebacate, Polyglyceryl-2 Diisostearate, Polyglyceryl-2 Dipolyhydroxystearate, PEG-30 Dipolyhydroxystearate, Glyceryl Stearate, and combinations thereof.

9. The cosmetic oil-in-water emulsion of claim 1, wherein the film-forming polymer is selected from the group consisting of Acrylates/C12-22 Alkyl Methacrylate Copolymer, Acrylates Copolymer, and combinations thereof; and wherein the film-forming polymer is at a concentration of about 0.05% to about 5.0% by total weight of the cosmetic oil-in-water emulsion.

10. The cosmetic oil-in-water emulsion of claim 9, wherein the film-forming polymer is at a concentration of about 0.5% to about 2.0% by total weight of the cosmetic oil-in-water emulsion.

11. The cosmetic oil-in-water emulsion of claim 1, wherein the oil-in water-emulsifier is selected from the group consisting of: Laureth-4, Steareth-21, PEG-100 Stearate, PEG-40 Stearate, and combinations thereof.

12. The cosmetic oil-in-water emulsion of claim 11, wherein the oil-in water-emulsifier is at concentration of about 0.1% to about 10.0% by total weight of the cosmetic oil-in-water emulsion.

13. The cosmetic oil-in-water emulsion of claim 11, wherein the oil-in water-emulsifier is at a concentration of about 1.0% to about 5.0% by total weight of the cosmetic oil-in-water emulsion.

14. The cosmetic oil-in-water emulsion of claim 1, further comprising a skin protectant, the skin protectant being selected from the group consisting of: Allantoin, Dimethicone, and Zinc Oxide.

15. A cosmetic oil-in-water emulsion comprising:
water at a concentration of about 70% to about 95% by total weight of the cosmetic oil-in-water emulsion;
a base oil;
an oil-in-water emulsifier;
at least two water-in-oil emulsifiers that are selected from the group consisting of: Diisostearoyl Polyglyceryl-3 Dimer Dilinoleate, Polyglyceryl-4 Diisostearate/Polyhydroxystearate/Sebacate, Polyglyceryl-2 Diisostearate, Polyglyceryl-2 Dipolyhydroxystearate, PEG-30 Dipolyhydroxystearate, Glyceryl Stearate, and combinations thereof; and
a film forming polymer.

16. The cosmetic oil-in-water emulsion of claim 15, wherein the film forming polymer is selected from the group consisting of: Acrylates/C12-22 Alkyl Methacrylate Copolymer, Acrylates Copolymer, and combinations thereof, and wherein the film forming polymer is at a concentration of about 0.05% to about 5.0% by total weight of the cosmetic oil-in-water emulsion.

17. The cosmetic oil-in-water emulsion of claim 15, wherein the oil-in water-emulsifier is selected from the group consisting of: Laureth-4, Steareth-21, PEG-100 Stearate, PEG-40 Stearate, and a combination thereof.

18. The cosmetic oil-in-water emulsion of claim 15, wherein the base oil has a wVTR of about 5 to 30 g/m²/hr and is at a concentration of no more than about 15% by total weight of the cosmetic oil-in-water emulsion.

19. The cosmetic oil-in-water emulsion of claim 15, wherein the cosmetic oil-in-water emulsion has a post-immersion wVTR value of between 0 to about 40 g/m²/hr and an Oxygen Permeability of at least about 1.0 (cc/100 in² per day).

20. The emulsion of claim 15, wherein the cosmetic oil-in-water emulsion has a pre-immersion wVTR value of less than 30 g/m²/hr.
